(19) United States
(12) Patent Application Publication
(10) Pub. No.: US 2008/0207914 A1
(43) Pub. Date: Aug. 28, 2008

(54) BIS-(1(2) H-TETRAZOL-5-YL)-AMINE MONOHYDRATE SYNTHESIS AND REACTION INTERMEDIATE

(75) Inventors: Toshiyuki Toda, Takasago-shi (JP); Toru Kofukuda, Takasago-shi (JP)

Correspondence Address: MOTS LAW, PLLC, 1001 PENNSYLVANIA AVE. N.W., SOUTH, SUITE 600, WASHINGTON, DC, 20004, US

(73) Assignee: TOYO KASEI KOGYO CO., LTD., Takasago-shi (JP)

(21) Appl. No.: 12/038,938

(22) Filed: Feb. 28, 2008

Related U.S. Application Data
(60) Provisional application No. 60/904,189, filed on Mar. 1, 2007.

(30) Foreign Application Priority Data
Feb. 28, 2007 (JP) 2007-049949

(51) Int. Cl. C07D 255/02

(52) U.S. Cl. 548/251

(57) ABSTRACT

The present invention provides Bis-(1(2)H-tetrazol-5-YL)-amine compound, and safe and inexpensive methods for producing high quality Bis-(1(2)H-tetrazol-5-YL)-amine compound. Also provided are Bis-(1(2)H-tetrazol-5-YL)-amine intermediates, and methods for their preparation. The process of preparing Bis-(1(2)H-tetrazol-5-YL)-amine intermediate substantially combines dicyanamide salt, azide salt, solvent and acid at 40-70 C temperature. The process of preparing Bis-(1(2)H-tetrazol-5-YL)-amine anhydride and/or Bis-(1(2)H-tetrazol-5-YL)-amine monohydrate comprises heating the intermediate described above at ≧75 C and adding a second acid.

### CROSS REFERENCE TO RELATED APPLICATIONS

This application receives priority from U.S. patent application Ser. No. 60/904,189 filed on Mar. 1, 2007 under 35 USC 119, the contents of which are incorporated by reference.

### BACKGROUND OF THE INVENTION

1. Field of the Invention

The present invention relates to the preparation of Bis-(1(2)H-tetrazol-5-YL)-amine compound and its intermediates by the use of azide salt and dicyanamide salt.

2. Description of Related Art

Bis-(1(2)H-tetrazol-5-YL)-amine is a useful compound as a gas producing agent. The synthesis method using azide salt and dicyanamide salt is known and described in the Journal of Organic Chemistry (1964) Vol. 29. p 650-660. (abbreviated as JOC), WO brochure 95/18802, Japan patent application number 2004-67544, Japan patent application number 2004-323392, and US patent number 2003/0060634.

The method described in JOC syntheses Bis-(1(2)H-tetrazol-5-YL)-amine monohydrate by reaction with trimethylammoniun chloride as an acid in water under reflux conditions for 23 hours followed by HCl treatment (67% yield).

In the method described in the WO 95/18802 brochure, Bis-(1(2)H-tetrazol-5-YL)-amine (anhydride) is provided by reaction with a full excess of boric acid in water for 48 hours followed by HCl treatment (80.3% yield).

In the method described in Japanese patent application 2004-67544, Bis-(1(2)H-tetrazol-5-YL)-amine monohydrate is provided by reaction with an aprotic solvent, such as N,N-dimethylformamide, and ammonium halide as an acid, such as sodium ammonium at 100˜150 C for 5˜10 hours followed by HCl treatment (80˜90% yield).

The method described in Japanese patent application 2004-323392, reacts a protonic acid, such as HCl, and manganese chloride as a metal chloride compound in water or aqueous solvent at 95˜105 C for 24 hours. Metal salt is filtered, treated with a protonic acid such as HCl, and generates Bis-(1(2)H-tetrazol-5-YL)-amine monohydrate (yield 80˜90%)

The method described in US patent 20030060634, maintains hydrazoic acid in the reactor, by adding an acid of pKa <2 slowly to the solution (generally over 12-24 hours) controlling pH relatively high, at 65 C temperature (preferably under reflux condition). After the reaction, treatment with a protonic acid such as HCl generates Bis-(1(2)H-tetrazol-5-YL)-amine monohydrate. (yield 86%)

The methods above have several disadvantages listed below.

The method described in JOC needs trimethylammonium, which is expensive and may produce an explosive sublimate compound, triammonium azide.

The method described in WO 95/18802 brochure has a long reaction time of 48 hours, and uses boric acid, which is hard to remove by decomposition.

The method described in Japanese patent application 2004-67544 uses organic solvent, which is costly to both purchase and dispose. Furthermore, this method may produce an explosive sublimate compound, ammonium azide.

The method described in Japanese patent application 2004-323392 uses manganese salt, which may complicate post treatment and it makes total synthesis cost high.

The method described in U.S. patent number 20030060634 needs a lengthy and expensive step of adding a strong acid dropwise. This high temperature step increases the concentration of hydrogen azide gas and effects the quality of the final product. Furthermore, the final compound is produced only as monohydrate crystals. To make anhydride product, a higher temperature is needed. In addition, this anhydride compound becomes monohydrate compound immediately at ≧10% humidity.

In consideration of these issues, the present invention aims to provide a method that is safe and that can produce these compounds in high quality at low cost.

### SUMMARY OF THE INVENTION

The invented process solves the above issues and can produce Bis-(1(2)H-tetrazol-5-YL)-amine compound intermediate substantially by combining dicyanamide salt, azide salt, solvent, and acid at 40-70 C temperature.

In the process of the present invention, alkali metal salt, alkaline earth metal salt, and ammonium salt can be used as a dicyanamide salt. Sodium dicyanamide is most preferable.

As for an azide salt, alkali metal salt and alkaline earth metal salt can be used. Sodium azide is most preferred. And the mole ratio of this azide salt to dicyanamide is 1.90-2.2. In particular, the mole ratio of 2.0-2.1 mole is especially preferable.

As for a solvent, water and water soluble or miscible solvents such as an aprotic polar solvent, N-substituted lactam solvent, alcohol, ether, or mixture of these can be used. A suitable aprotic polar solvent is N,N-dimethylformamide, N,N-diethylformamide, N,N-diisopropylformamide, N,N-dimethylacetoamide, N,N-diisopropylacetamide, N,N-dimethylpropionacetamide, N,N-diethylpropionacetoamide, and so on. A suitable N-substituted lactam solvent is N-methylpyrrolidon, N-ethylpyrolidon or so on. A suitable alcohol is methanol, ethanol, isopropyl alcohol, and so on. A suitable ether is tetrahydrofuran, dioxane, methyl cellosolve(ethyleneglycol monomethyl ether), ethyl cellosolve(ethylene glycol monoethyl ether) and so on. The solvent can be chosen from among the above or mixtures thereof. Water is a most preferable solvent considering the cost. In this case, another solvent, which is soluble with or miscible with water, can be added. As for the amount of the solvent, a weight ratio of solvent to dicyanamide of 1-100, and especially 5-20 is preferable.

As for an acid, hydrochloric acid (HCl), sulfuric acid, nitric acid, phosphoric acid, and perchloric acid, which is inorganic acid, also formic acid, acetic acid, propionic acid, methanesulfonic acid, toluenesulfonic acid, trifluoroacetic acid, and trifluoromethanesulfonic acid, which is organic acid can be used. Considering cost for all process, purchasing, and industrial view points, HCl, sulfuric acid, or glacial acetic acid are preferable, and because of reusability, HCl is the best.

As for the amount of the acid, the mole ratio of the acid to dicyanamide is 1.0-2.0 mole, especially 1.1-1.9 mole per one mole of dicyanamide is preferable.

The order is not critical for the mixture as long as the four individual ingredients of dicyanamide salt, azide salt, solvent and acid are combined.

For instance, the acid can be added to a mixture of the dicyanamide salt and the azide salt in the solvent. In this case, the acid should be added over a time period of 30 minutes to 4 hours at 15-70 C temperature. Similarly, the azide salt and the acid mixture can be added to dicyanamide solution, the mixture of the dicyanamide and the solvent. In this case, the mixture should be added over 30 minutes-12 hours at 40-70 C temperature. The acid can be added at once at constant temperature, and half can be added at low temperature followed by heating and addition of the remaining half. Alternatively, the dicyanamide solution and the acid can be mixed gradually in small amounts.

This mix should be done by stirring vigorously. As for the correlation between temperature and time, a high temperature is dangerous, and needs more time for mixing. Besides, a low temperature makes mixing easier and shortens the time. In this respect, low temperature seems preferable. But low temperature decreases the reaction speed of intermediates, which then requires more acid, and may cause a lower yield. Conversely, high temperature will speed the reaction of intermediates, but may cause formation of side product contaminants and long mixing times.

Therefore, temperature and the time were determined by prioritizing factors such as process time, safety, yield, and purity.

Synthesizing intermediates by adding acid to dicyanamide salt and azide salt solution at low temperature can reduce the concentration of azide hydrogen even when followed by a ring closure reaction at high temperature.

When adding a strong acid such as HCl or sulfuric acid, the lower concentration of the acid, and vigorous stirring during addition are preferable.

‘Preparation of substantial intermediate’ means that the total yield of intermediate is 70% or more and Bis-(1(2)H-tetrazol-5-YL)-amine compound is 5% or less. At this point, the remaining dicyanamide salt does not matter since it will be changed into an intermediate during the next heating step by acid derived from Bis-(1(2)H-tetrazol-5-YL)-amine compound synthesis. Too much Bis-(1(2)H-tetrazol-5-YL)-amine compound means that the reaction temperature is too high. In this case, side compounds formed of Bis-(1(2)H-tetrazol-5-YL)-amine compound will increase during the following steps.

The present invention solved the above issues, and can produce Bis-(1(2)H-tetrazol-5-YL)-amine anhydride or Bis-(1(2)H-tetrazol-5-YL)-amine monohydrate by heating the intermediates prepared above.

The heating reaction should be carried out at 75 C or at a higher temperature, preferably ≧90 C for a time period of for 10-48 hours, preferably 10-30 hours. If the temperature is ≦75 C, then this condition may cause a long reaction time, since the reaction speed becomes slow and the reaction cannot be completed. The heating reaction can be performed either under ambient pressure or elevated pressure.

As for the second acid solution, the same acid should be used as the first acid, which is selected for the process of preparing the intermediate.

This second acid can be added during the above intermediate heating step at 75 C, or after a cool down following that treatment.

At the time of adding acid, by controlling the intermediate temperature, Bis-(1(2)H-tetrazol-5-YL)-amine monohydrate and Bis-(1(2)H-tetrazol-5-YL)-amine anhydride can be produced selectively. That is to say, regulation of the intermediate temperature less than 70 C can produce Bis-(1(2)H-tetrazol-5-YL)-amine monohydrate mainly. A temperature of 70 C or higher can produce primarily Bis-(1(2)H-tetrazol-5-YL)-amine anhydride. This second acid is added for over 2 hours, and preferably 4-12 hours by drop, to complete the reaction in a short time. To complete the reaction and increase yield, acid should be added until the pH drops to 3 or less.

After the reaction, by controlling pH with added amine, Bis-(1(2)H-tetrazol-5-YL)-amine salt can be produced.

As described above, the present invention shows that combining dicyanamide salt, azide salt, solvent, and acid at 40-70 C, and at relatively low temperature can safely yield the intermediates. Additionally, after the preparation of intermediates, the reaction can proceed to completion by heating at ≧75 C temperature and adding a second acid, to allow the safe production of high quality Bis-(1(2)H-tetrazol-5-YL)-amine compound in a high yield and in a short time.

### DETAILED DESCRIPTION OF EMBODIMENTS

Below are embodiments that show the invention in detail. The invention is not limited in these embodiments, but may apply to various alterations.

The Process of Bis-(1(2)H-tetrazol-5-YL)-amine Intermediate Preparation:

Dissolve dicyanamide salt, azide salt (mole ratio to dicyanamide is 2.0-2.1 to 1) in water (volume ratio to dicyanamide is 5-10 to 1). Keep the mixture at 40-60 C and add acid for over 2-12 hours while mixing vigorously. This results in preparation of intermediate.

The process of Bis-(1(2)H-tetrazol-5-YL)-amine Monohydrate Preparation:

Dissolve dicyanamide salt and azide salt (mole ratio to dicyanamide is 2.0-2.1 to 1) in water such that the volume ratio to dicyanamide is 5-10 to 1. Maintain the mixture at 40-60 C and add acid (mole ratio to dicyanamide is 1.1-1.9 to 1) for over 2-12 hours with mixing vigorously. Mix the solution at ≧90 C until reaction is complete. This generally takes 12-48 hours. Add acid gradually until pH of ≦2 at ≦60 C. After reaction, cool the mixture to around room temperature, filter crystals, and recover Bis-(1(2)H-tetrazol-5-YL)-amine monohydrate (yield 85-95%).

The Process of Bis-(1(2)H-tetrazol-5-YL)-amine Anhydride Preparation:

Dissolve dicyanamide salt and azide salt (mole ratio to dicyanamide is 2.0-2.1 to 1) in water (volume ratio to dicyanamide is 5-10 to 1). Keep the mixture at 40-60 C and add acid (mole ratio to dicyanamide is 1.1-1.9 to 1) for over 2-12 hours while mixing vigorously. Mix the solution at ≧90 C for 6-24 hours (depending on the reaction temperature) until the intermediate becomes 5-20%. Add acid until the pH becomes ≦3 at ≧80 C. Cool the mixture around room temperature, and filter the crystals, to recover Bis-(1(2)H-tetrazol-5-YL)-amine anhydride (yield 85-95%).

The Process of Bis-(1(2)H-tetrazol-5-YL)-amine Monohydrate Preparation:

Dissolve azide salt (mole ratio to dicyanamide is 2.0-2.1 to 1) in water (volume ratio to dicyanamide is 5-10 to 1). Keep the mixture cool and add acid (mole ratio to dicyanamide is 1.1-1.9 to 1) to make azide hydro solution. Keep the mixture at 40-60 C and add this solution as acid (mole ratio of acid to dicyanamide is 1.1-1.9 to 1) to dicyanamide solution for over 2-12 hours while mixing vigorously. Mix the solution at ≧90 C until reaction is complete (generally takes 12-48 hours). Add acid until pH becomes ≦3 at ≦60 C. Cool the mixture to around room temperature, and filter the crystals, to recover Bis-(1(2)H-tetrazol-5-YL)-amine monohydrate (yield 85-95%).

The Process of Bis-(1(2)H-tetrazol-5-YL)-amine Monoammonium Salt Preparation:

Dissolve dicyanamide salt, azide salt (mole ratio to dicyanamide is 2.0-2.1 to 1) in water (volume ratio to dicyanamide is 5-10 to 1). Keep the mixture at 40-60 C and add acid (mole ratio to dicyanamide is 1.1-1.9 to 1) for over 2-12 hours while mixing vigorously. Mix the solution at ≧90 C until reaction is complete (generally takes 12-48 hours), and add acid gradually until pH of ≦2 at ≦60 C. Add ammonia until the pH becomes around 4.5. Cool the mixture around room temperature, and filter crystals to recover Bis-(1(2)H-tetrazol-5-YL)-amine mono ammonium salt (yield 80-90%).

### Case Example 1

### Synthesis of Bis-(1(2)H-tetrazol-5-YL)-amine Intermediate

Sodium dicyanamide (NaDCA) 13.35 g (0.15 mol), sodium azide 19.50 g (0.30 mol), and water 100 mL were combined in a 200 mL flask, heated to 60 C, and 25% of HCl 39.4 g (0.27 mol) added dropwise over 4 hours while stirring vigorously. The yield was 90%, including 1% of NaDCA, 87% of 3 intermediates (10, 2, and 75% respectively), and 1% of Bis-(1(2)H-tetrazol-5-YL)-amine compound.

### Case Example 2

### Synthesis of Bis-(1(2)H-tetrazol-5-YL)-amine Monohydrate

Sodium dicyanamide (NaDCA) 13.35 g (0.15 mol), sodium azide 19.50 g (0.30 mol), and water 100 mL were combined in 200 mL flask, heated to 60 C, 25% of HCl 39.4 g (0.27 mol) was added dropwise over 4 hours while stirring vigorously. After 24 hours of reflux, this was cooled to 50 C, HCl conc. 29.0 g added, and pH of ≦2 checked. This was cooled until 20 C and the mixture was filtered and washed with an excess of water. Vacuum drying at 60 C provided white crystals of Bis-(1(2)H-tetrazol-5-YL)-amine monohydrate 23.27 g. (Yield 90.7%, HPLC purity 99.4%)

### Case Example 3

### Synthesis of Bis-(1(2)H-tetrazol-5-YL)-amine Monohydrate

NaDCA 17.81 g (0.20 mol), and water 50 ml were prepared in a 200 mL flask.

To another flask, while cooling in an ice bath, sodium azide 26.26 g (0.40 mol) and water 100 mL were combined. Concentrated HCl 25.0 g (0.24 mol) was added dropwise. NaDCA solution was heated to 60 C, and cooled azide hydro solution was added dropwise over 2 hours. After 24 hours of reflux, this was cooled to 50 C, and HCl conc. 39.6 g was added while checking the pH of ≦2. This was cooled down to 20 C and the mixture filtered and washed by an excess of water. Vacuum drying at 60 C provided white crystals of Bis-(1(2)H-tetrazol-5-YL)-amine monohydrate 31.37 g. (Yield 91.7%, HPLC purity 99.2%)

### Case Example 4

### Synthesis of Bis-(1(2)H-tetrazol-5-YL)-amine Monohydrate

NaDCA 17.81 g (0.20 mol), and water 50 ml were prepared in a 200 mL flask. To another flask, with cooling in an ice bath, sodium azide 26.26 g (0.40 mol) and water 100 mL were combined. Concentrated HCl 25.0 g (0.24 mol) was added dropwise. NaDCA solution was heated to 60 C, and cooled azide hydro solution was added dropwise over 2 hours. After 24 hours of reflux, this was cooled to 50 C, HCl conc. 39.6 g was added, and pH checked at ≦2. This was cooled to 20 C and filtered and washed by an excess of water. Vacuum drying at 60 C provided white crystals of Bis-(1(2)H-tetrazol-5-YL)-amine monohydrate 31.37 g. (Yield 91.7%, HPLC purity 99.2%)

### Case Example 5

### Synthesis of Bis-(1(2)H-tetrazol-5-YL)-amine Monohydrate

NaDCA 19.00 g (0.21 mol), sodium azide 27.70 g (0.42 mol), and water 115 mL were combined in a 200 mL flask and heated to 60 C Acetic acid 24.35 g (0.40 mol) was added dropwise over 4 hours. After 24 hours of reflux, this was cooled to 50 C, HCl conc. 67.6 g was added, and pH checked at ≦2. This was cooled to 20 C and filtered to get crystals, which were washed by an excess of water. This was vacuum dried at 60 C to obtain white crystals of Bis-(1(2)H-tetrazol-5-YL)-amine monohydrate 32.55 g. (Yield 89.1%, HPLC purity 99.0%)

### Case Example 6

### Synthesis of Bis-(1(2)H-tetrazol-5-YL)-amine Anhydride

NaDCA 17.81 g (0.20 mol), sodium azide 26.00 g (0.40 mol), and water 100 ml were combined in a 200 mL flask, and heated to 60 C HCl conc. 39.6 g (0.38 mol) was added dropwise over 4 hours with stirring vigorously. After 12 hours of reflux, HCl conc. 27.1 g was added dropwise over 3 hours at 75 C, and pH checked at ≦2. This was cooled to 20 C and filtered to get crystals, which were washed by an excess of water. This was vacuum dried at 60 C to obtain white crystals of Bis-(1(2)H-tetrazol-5-YL)-amine anhydride 27.11 g. (Yield 88.5%, HPLC purity 99.6%)

### Case Example 7

### Synthesis of Bis-(1(2)H-tetrazol-5-YL)-amine Anhydride

NaDCA 17.81 g (0.20 mol), sodium azide 26.00 g (0.40 mol), N,N-dimethylformamide 9 mL and water 100 ml were combined in a 200 mL flask and heated to 60 C. HCl conc. 39.6 g (0.38 mol) was added dropwise over 4 hours with stirring vigorously.

After 24 hours of reflux, this was cooled to 50 C, HCl conc. 67.6 g was added, and checked for a pH of ≦2. This was cooled until 20 C and filtered to get crystals, which were washed by an excess of water. This was vacuum dried at 60 C to obtain white crystals of Bis-(1(2)H-tetrazol-5-YL)-amine anhydride 28.30 g. (Yield 92.4%, HPLC purity 99.2%)

### Case Example 8

### Synthesis of Bis-(1(2)H-tetrazol-5-YL)-amine Monoammonium

NaDCA 17.81 g (0.20 mol), sodium azide 26.00 g (0.40 mol), and water 100 ml were combined in a 200 mL flask, and heated to 60 C. HCl conc. 39.37 g (0.27 mol) was added dropwise over 4 hours with stirring vigorously. After 24 hours of reflux, this was cooled to 50 C, HCl conc. 29.0 g, was added and checked pH for ≦2. Then ammonium was added gradually until pH reached 4.5. This was cooled until 20 C and filtered to obtain crystals, which were washed by an excess of water. Vacuum drying at 60 C obtained white crystals of Bis-(1(2)H-tetrazol-5-YL)-amine monoammonium 29.77 g. (Yield 87.5%, HPLC purity 99.4%)

### Case Example 9

### Synthesis of Bis-(1(2)H-tetrazol-5-YL)-amine Monohydrate

NaDCA 17.81 g (0.20 mol), sodium azide 26.00 g (0.40 mol), and water 170 ml were combined in a 200 mL flask. The solution was kept at 15 C and 20% of sulfuric acid 38.90 g (0.08 mol) was added dropwise over 2 hours with stirring vigorously. After 24 hours of reflux, this was cooled to 50 C, 60% of sulfuric acid 24.7 g was added by drop, and checked pH for ≦2. This was cooled to 20 C and filtered to obtain crystals, which were washed by an excess of water. Vacuum drying at 60 C obtained white crystals of Bis-(1(2)H-tetrazol-5-YL)-amine monohydrate 29.43 g. (Yield 86.0%, HPLC purity 98.3%)

As described above, products can be used as Bis-(1(2)H-tetrazol-5-YL)-amine compound of good quality that are valuable as a gassing agent necessary for airbag, or as a foaming agent. The invention includes application of these described methods to a wide variety of conditions and embodiments. Therefore, the scope of the invention is not limited by these embodiments, but instead defined by the claims which relate to and can be explained by the specification embodiments.

What is claimed is:

1. A process of preparing Bis-(1(2)H-tetrazol-5-YL)-amine intermediate characterized by combining dicyanamide salt, azide salt, solvent, and acid at 15-70 C temperature.

2. The process of claim 1 wherein said acid is added over a 30 minute to 4 hour period at 15-70 C temperature to the mixture of dicyanamide salt and azide salt.

3. The process of claim 1 wherein said azide salt and acid mixture are added to dicyanamide solution over a 30 minute to 4 hour period at 15-70 C temperature.

4. The process of claim 1 wherein said temperature is maintained at less than 65 C for the entire process.

5. The process of claim 1 wherein the mole ratio of azide salt to dicyanamide salt is 1.90-2.20 to 1.

6. The process of claim 1 wherein the mole ratio of acid to dicyanamide salt is 1.0-2.0 to 1.

7. The process of claim 1 wherein said dicyanamide salt is an alkaline metal salt or an alkaline earth metal salt.

8. The process of claim 1 wherein said azide salt is an alkaline metal salt or an alkaline earth metal salt.

9. The process of claim 1 wherein said solvent is water.

10. The process of claim 1 wherein said solvent is a combination of water and another solvent selected from the group consisting of non-protonic polar solvent, N-substitution lactan solvent, alcohol, and ether.

11. Bis-(1(2)H-tetrazol-5-YL)-amine intermediate produced by the method of claim 1, wherein Bis-(1(2)H-tetrazol-5-YL)-amine is 5% or less.

12. A process for making Bis-(1(2)H-tetrazol-5-YL)-amine anhydride or Bis-(1(2)H-tetrazol-5-YL)-amine monohydrate, comprising heating one or more intermediates at 75 C or higher, and adding a second acid.

13. The process of claim 12 wherein said intermediate is treated at 75 C or higher for 10-30 hours.

14. The process of claim 12 wherein said second acid is added during the heating of one or more intermediates at 75 C or higher.

15. The process of claim 12 wherein said controlled high temperature during addition of the second acid produces anhydrate or monohydrate compound selectively.

16. The process of claim 15 wherein the temperature when adding a second acid at less than 70 C, can produce Bis-(1(2)H-tetrazol-5-YL)-amine monohydrate compound selectively.

17. The process of claim 15 wherein the temperature when adding second acid at 70 C or more can produce Bis-(1(2)H-tetrazol-5-YL)-amine anhydride compound selectively.

18. The process of claim 12 wherein the period of time for adding acid is 2-12 hours.

19. The process of claim 12 wherein the second acid is added until the mixture becomes pH 3.

20. At least one of Bis-(1(2)H-tetrazol-5-YL)-amine anhydride and Bis-(1(2)H-tetrazol-5-YL)-amine monohydrate compound produced by the process of claim 12.

21. The process of preparing Bis-(1(2)H-tetrazol-5-YL)-amine salt comprising adding amine to at least one of Bis-(1(2)H-tetrazol-5-YL)-amine anhydride and Bis-(1(2)H-tetrazol-5-YL)-amine monohydrate produced by claim 20.

22. Bis-(1(2)H-tetrazol-5-YL)-amine salt produced by the process of claim 21.
